(19) United States
(12) Patent Application Publication
(10) Pub. No.: US 2022/0047740 A1
(43) Pub. Date: Feb. 17, 2022

(54) ULTRAVIOLET LIGHT STERILIZER, DISPENSER AND STORAGE FOR ITEMS

(71) Applicants: Bradley Scott MAXFIELD, Mapleton, UT (US); Christian David MILLS David MILLS, Highland, UT (US); Alex William ENCE, Mapleton, UT (US)

(72) Inventors: Bradley Scott MAXFIELD, Mapleton, UT (US); Christian David MILLS David MILLS, Highland, UT (US); Alex William ENCE, Mapleton, UT (US)

(21) Appl. No.: 17/401,074

(22) Filed: Aug. 12, 2021

Related U.S. Application Data
(60) Provisional application No. 63/064,846, filed on Aug. 12, 2020.

(51) Int. Cl. A61L 2/10

(52) U.S. Cl. CPC A61L 2/10; A61L 2202/11; A61L 2202/121; A61L 2202/14; A61L 2202/122

(57) ABSTRACT

Embodiments of the invention disclosed herein include devices for sterilizing items, particularly hand-held writing implements, using ultraviolet radiation. Other useful features of the sterilizer embodiments include a no touch proximity sensor for opening the lid to access sterilization ports and any items within the ports, automatic descent of the items into the sterilizer and closure of the lid for a preselected sterilization cycle without the lid binding on the items in the ports.

### CROSS REFERENCE TO APPLICATION

The present invention claims the benefit of priority from U.S. Provisional Application 63/064,846, filed on Aug. 12, 2020, entitled “ULTRAVIOLET LIGHT STERILIZER, DISPENSER AND STORAGE FOR ITEMS” and incorporates it in its entirety by this reference.

### BACKGROUND OF THE INVENTION

### Field of the Invention

The present invention relates generally to sterilization devices. More particularly, this invention relates to a device for sterilizing items of similar size, such as hand-held writing implements or other objects of similar size using ultraviolet (UV) radiation.

### Description of Related Art

It is well known that germs are spread by hand to hand contact. There have been many efforts to address this problem, starting with regular hand washing. Hand sanitizers are also commonly used and do not require a source of water or means to dry hands afterward.

Germs can also be transmitted to objects such as toothbrushes and writing instruments. One approach to killing germs or bacteria on objects employs the use of ultraviolet light. Using UV light as a germicide and sterilizer has been applied in the context of toothbrushes and dental implements, for example, see U.S. Pat. No. 3,954,407 to Andary et al., U.S. Pat. No. 4,772,795 to Sakurai et al., U.S. Pat. No. 4,803,364 to Ritter, U.S. Pat. No. 4,806,770 to Hylton et al., U.S. Pat. No. 4,906,851 to Beasley et al., U.S. Pat. No. 4,973,847 to Lackey et al., U.S. Pat. No. 5,023,460 to Foster, Jr. et al., U.S. Pat. No. 5,126,572 to Chu, U.S. Pat. No. 5,127,521 to Bourque, U.S. Pat. No. 5,487,877 to Choi and U.S. Pat. No. 5,547,635 to Duthie, Jr.

Ultraviolet light has also been applied in the context of sterilizing writing implements, for example, see U.S. Pat. No. 6,039,928 to Roberts, U.S. Pat. No. 7,642,524 to Alvarez et al., U.S. Pat. No. 8,058,629 to Long, U.S. Pat. No. 8,337,770 to Wind, U.S. Pat. No. 9,345,799 to Wind, U.S. Pat. No. 9,511,160 to Wind and U.S. Pat. No. 9,566,819 to McDowell. As the germicidal effects of using UV light are well known from the prior art listed above and to one of ordinary skill in the art, it will not be further explained herein.

While these prior art devices are capable of providing sterilization capability for various objects, it is not readily apparent that these devices are intuitive to use, along with easy loading, unloading and dispensing of writing implements or other objects that are of the same general size and shape. Accordingly, there exists a need in the art for an ultraviolet light sterilizer, dispenser and storage for similarly sized items, such as writing implements.

### BRIEF SUMMARY OF THE INVENTION

Embodiments of the invention include sterilization devices for hand-held items. The hand-held items may be, for example and not by way of limitation, writing implements, such as pens, pencils, markers and highlighters.

Additional features and advantages of the invention will be apparent from the detailed description which follows, taken in conjunction with the accompanying drawings, which together illustrate, by way of example, features of embodiments of the present invention.

### BRIEF DESCRIPTION OF THE SEVERAL VIEWS OF THE DRAWINGS

The following drawings illustrate exemplary embodiments for carrying out the invention. Like reference numerals refer to like parts in different views or embodiments of the present invention in the drawings.

FIGS. 1A and 1B are perspective views in image and line drawing, respectively, of an enclosed sterilizer, according to an embodiment of the present invention.

FIGS. 2A and 2B are perspective views of the embodiment of a sterilizer shown in FIGS. 1A and 1B with cylindrical covering transparent and removed, respectively.

FIGS. 3A and 3B are perspective image and line drawing views of the embodiment of a sterilizer shown in FIGS. 1A and 1B with lid open and writing instruments presented in sterilization ports.

FIGS. 4A and 4B are perspective image and line drawing views, respectively, of the embodiment of a sterilizer shown in FIGS. 1A and 1B with lid open showing sterilization ports.

FIGS. 5A and 5B are perspective transparent image and line drawing views, respectively, of the embodiment of a sterilizer shown in FIGS. 4A and 4B with lid open showing sterilization ports and internal features.

FIGS. 6A and 6B are side image and line drawing views, respectively, of the embodiment of a sterilizer shown in FIGS. 1A and 1B.

FIGS. 7A and 7B are side transparent image and line drawing views, respectively, of the embodiment of a sterilizer shown in FIGS. 6A and 6B.

FIGS. 8A and 8B are side image and line drawing views, respectively, of the embodiment of a sterilizer shown in FIGS. 4A and 4B.

FIGS. 9A and 9B are side transparent image and cylindrical cover removed line drawing views, respectively, of the embodiment of a sterilizer shown in FIGS. 8A and 8B, illustrating internal features.

FIGS. 10A and 10B are side views of the embodiment of a sterilizer shown in FIGS. 3A and 3B, with cylindrical cover installed and cylindrical cover removed, respectively.

FIG. 11 is an enlarged side view of the embodiment of a sterilizer shown in FIG. 10B.

FIG. 12 is a perspective view of another embodiment of a sterilizer, according to the present invention.

FIG. 13 is a side transparent view of the embodiment of the sterilizer shown in FIG. 12, with lid open illustrating internal features.

FIG. 14 is a perspective view of yet another embodiment of a sterilizer, according to the present invention.

FIG. 15 is a cross-sectional perspective view the embodiment of the sterilizer shown in FIG. 14.

FIG. 16 is another side image view of the embodiment of the sterilizer shown in FIGS. 10B and 11.

### DETAILED DESCRIPTION OF THE INVENTION

The embodiments of the invention disclosed herein are devices for sterilizing objects, particularly writing implements, using ultraviolet radiation. While the illustrated embodiments that show objects suitable for sterilization with the invention are writing implements, namely pens, it will be understood that other types of writing implements, such as pencils, highlighters, fountain pens, or even erasers, tooth brushes or dental implements may also be sterilized using the devices disclosed herein. Accordingly, the exemplary embodiments disclosed herein will be discussed in the context of a writing implement or “pen” for convenience of discussion. However, it will be understood that other objects suitable for sterilizing could, if suitably sized, be used with the present invention. Note further that by simply scaling the invention for a particular application, any suitable object(s) could be sterilized, stored and presented in essentially the same manner as disclosed herein.

The sterilizers disclosed herein could be used anywhere that people need access to germ free writing implements or utensils. Some locations where such a sterilizer could be used would be waiting rooms in hospitals, urgent care facilities, emergency rooms, doctor's offices, dentist's offices, pharmacies, chiropractors, etc. It is not uncommon for sick patients to use a public pen to fill out paperwork, forms and the like, required for the intended service provided. Embodiments of the sterilizer will provide a person with a sterile writing utensil without the fear of sharing germs with another potentially sick person. In such an environment, a pen might be used over and over by various patients and, thus, are likely to have been touched by hands that are not germ free. So, use of the sterilizers disclosed herein could help prevent the spread of disease. It will be understood that such a sterilizer as disclosed herein could also be used at nurse's stations, to prevent care providers from spreading disease between patients. It is well known that hospital acquired infections are common, dangerous, and expensive.

Potential target audiences or customers who would purchase embodiments of the sterilizer as disclosed herein may include, for example and not by way of limitation, hospital administrators or office managers who would purchase a sterilizer for use in their office facilities. Anticipated users of the embodiments of the sterilizer as disclosed herein may include anyone using a writing implement in a public place, where the writing implement is intended to be shared and used by more than one user.

Particularly useful features of the embodiments of the sterilizer disclosed herein include: (1) an automated mechanism for sterilizing one or more writing implements, (2) the capability of presenting or displaying a plurality of writing implements after they have been sterilized, and (3) safely containing the emitted ultraviolet light within the sterilizer during sterilization.

Another useful feature of an embodiment of the sterilizer disclosed herein includes a “no touch” proximity sensor near the lid that automatically senses a hand or fingers near the lid which opens the lid without having to touch the sterilizer. Building on the “no touch” opening feature is an automated mechanism for elevating the writing implements and presenting them to the user for use.

The plurality of sterilization ports within the various embodiments of a sterilizer allow for variance in size/shape and length of writing implements, for example from small pens to larger highlighters. All the writing implements automatically lower into the sterilizer once placed in at least one port and are automatically sterilized for a pre-selected amount of time by exposure to ultraviolet (UV) light. The writing implements automatically rise up and the lid opens upon completion of a sterilization cycle or if interrupted by proximity sensing. Each sterilization port accessed under the lid of the sterilizer leads to an individual quartz tube for holding, storing and dispensing the writing implement.

Additional features include a loose cable used in conjunction with the lid and the writing implement transport mechanism allows for the writing implements to descend before the lid closes, thereby preventing the lid from binding or closing down on the tops of the writing implements. A torsion spring is used to open the lid. The lid is intended to automatically open upon sterilization completion or proximity sensing. But, another useful feature is an extension spring that prevents the lid from breaking when the lid is pried open. The inner surface of the cylindrical cover of the sterilizer is a highly reflective surface to scatter the UV light within the sterilizer to expose every feature of the writing implements within their quartz tubes. According to one embodiment, a highly polished stainless steel inner surface of the cylindrical cover of the sterilizer is used to reflect the UV rays emanating from the UV lamp(s) within.

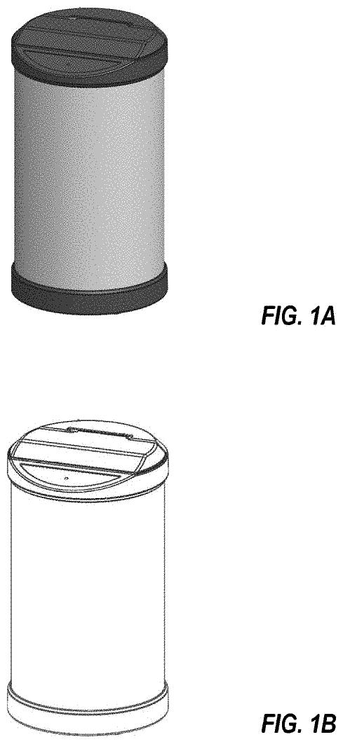

The detailed description will now focus on the various drawings disclosed herein. FIGS. 1A and 1B are perspective views in image and line drawing, respectively, of an enclosed sterilizer, according to an embodiment of the present invention.

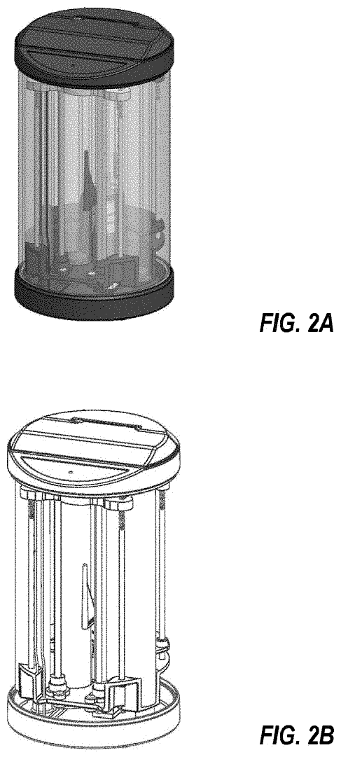

FIGS. 2A and 2B are perspective views of the embodiment of a sterilizer shown in FIGS. 1A and 1B with cylindrical covering transparent and removed, respectively. The lamps, elevator mechanism internal to the sterilizer are shown in FIGS. 2A and 2B.

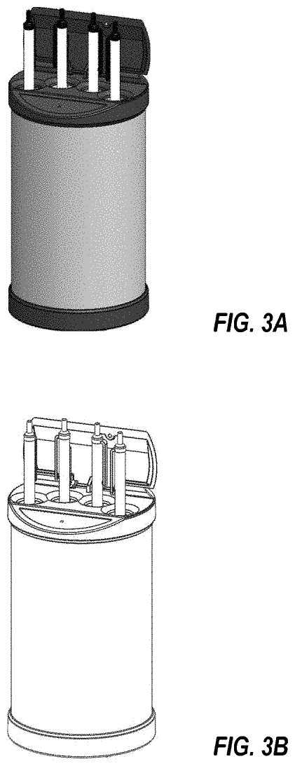

FIGS. 3A and 3B are perspective image and line drawing views of the embodiment of a sterilizer shown in FIGS. 1A and 1B with lid open and writing instruments presented in sterilization ports. As shown in FIGS. 3A and 3B, a plurality of items, i.e., four pens shown, may be displayed and dispensed from the open lid position.

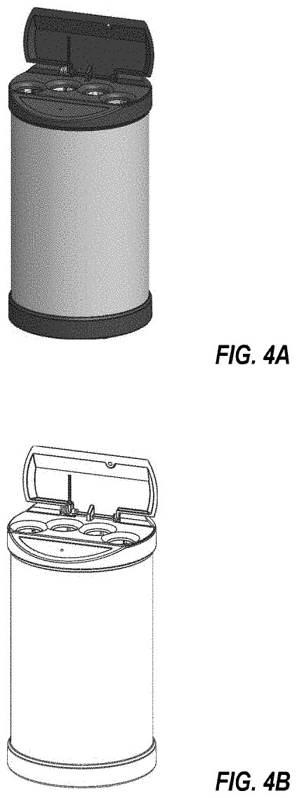

FIGS. 4A and 4B are perspective image and line drawing views, respectively, of the embodiment of a sterilizer shown in FIGS. 1A and 1B with lid open showing sterilization ports. FIGS. 4A and 4B are identical to FIGS. 3A and 3B with the pens removed, showing the open ports underneath the lid.

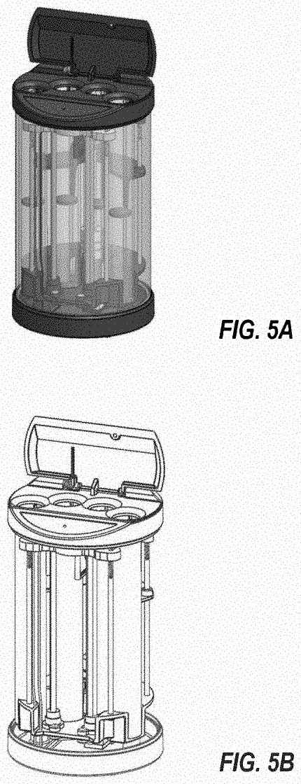

FIGS. 5A and 5B are perspective transparent image and line drawing views, respectively, of the embodiment of a sterilizer shown in FIGS. 4A and 4B with lid open showing sterilization ports and internal features.

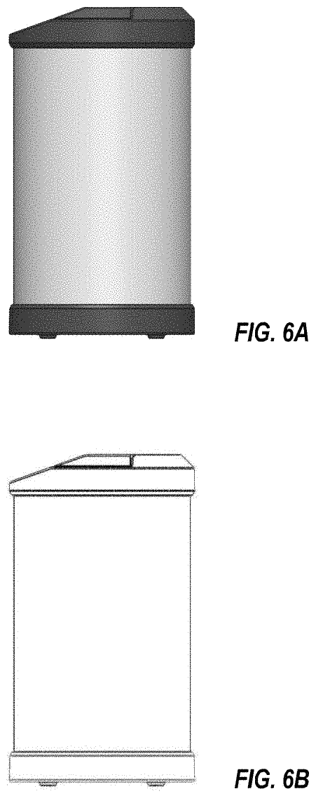

FIGS. 6A and 6B are side image and line drawing views, respectively, of the embodiment of a sterilizer shown in FIGS. 1A and 1B.

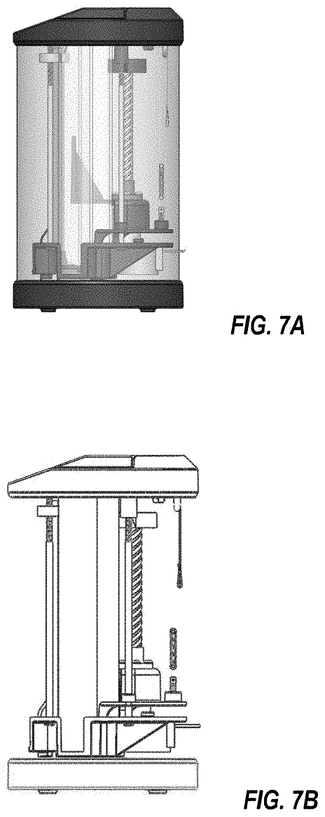

FIGS. 7A and 7B are side transparent image and line drawing views, respectively, of the embodiment of a sterilizer shown in FIGS. 6A and 6B.

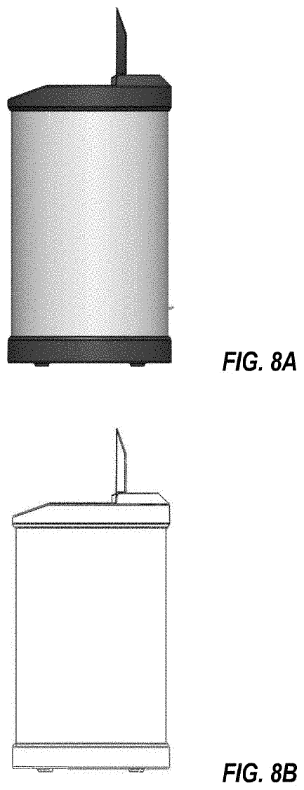

FIGS. 8A and 8B are side image and line drawing views, respectively, of the embodiment of a sterilizer shown in FIGS. 4A and 4B.

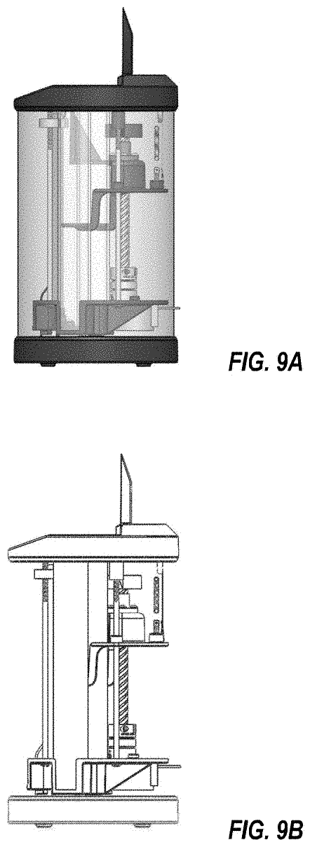

FIGS. 9A and 9B are side transparent image and cylindrical cover removed line drawing views, respectively, of the embodiment of a sterilizer shown in FIGS. 8A and 8B, illustrating internal features.

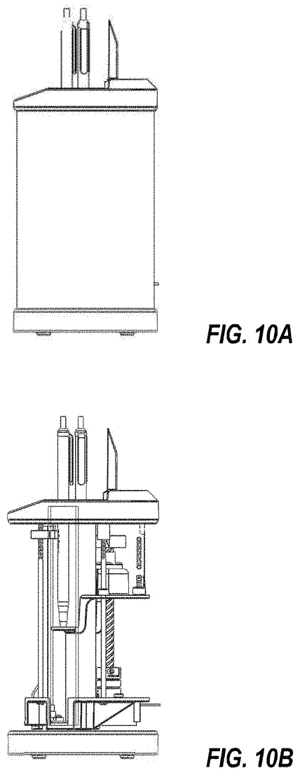

FIGS. 10A and 10B are side views of the embodiment of a sterilizer shown in FIGS. 3A and 3B, with cylindrical cover installed and cylindrical cover removed, respectively.

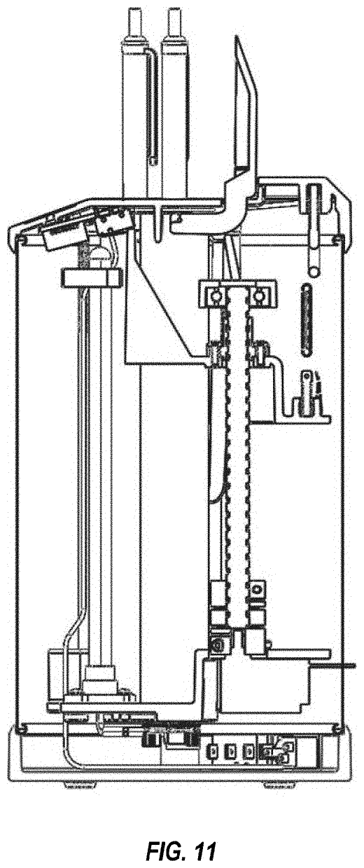

FIG. 11 is an enlarged side view of the embodiment of a sterilizer shown in FIG. 10B.

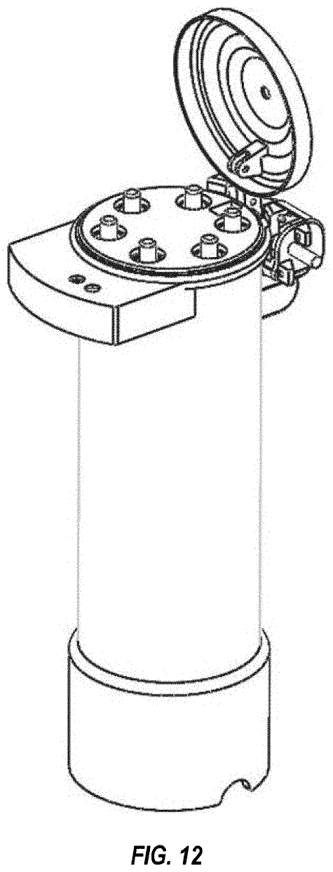

FIG. 12 is a perspective view of another embodiment of a sterilizer, according to the present invention. This particular embodiment elevates the items without a slot in the transparent tubes and thus may requires a taller body and cover. This particular embodiment also has a larger capacity, i.e., six items vs. four in the embodiment shown in FIGS. 1A to 11.

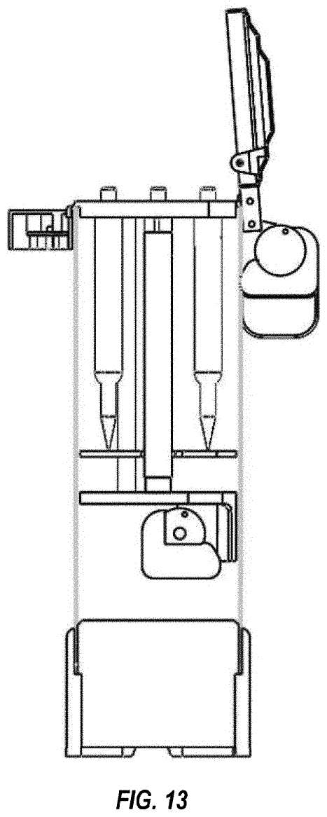

FIG. 13 is a side transparent view of the embodiment of the sterilizer shown in FIG. 12, with lid open illustrating internal features.

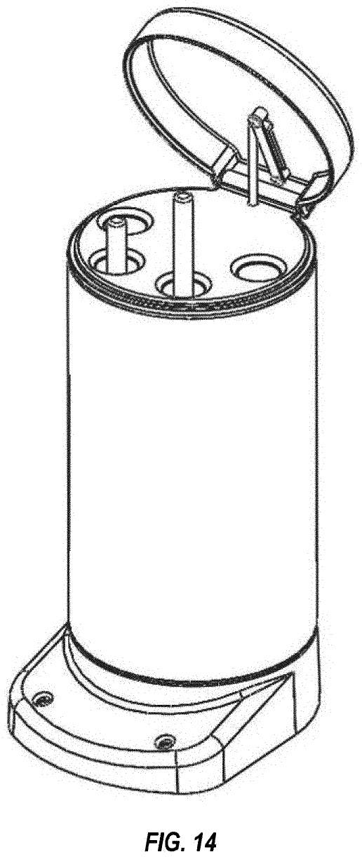

FIG. 14 is a perspective view of yet another embodiment of a sterilizer, according to the present invention. According to this particular embodiment, a capacity of four items is contemplated.

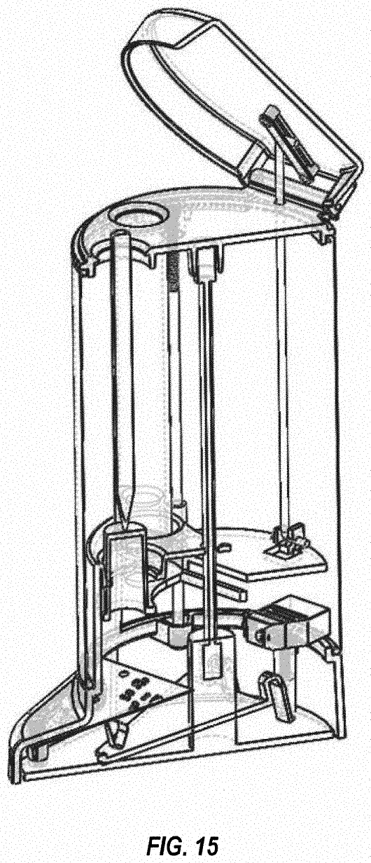

FIG. 15 is a cross-sectional perspective view the embodiment of the sterilizer shown in FIG. 14.

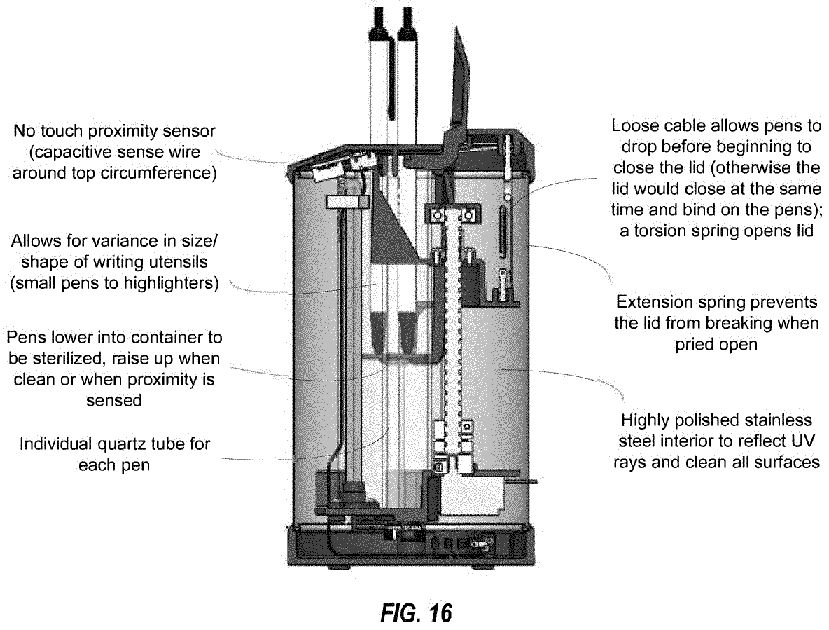

FIG. 16 is another side image view of the embodiment of the sterilizer shown in FIGS. 10B and 11. FIG. 16 illustrates a number of the novel features of this particular embodiment of a sterilizer. The sterilizer includes an embodiment of a no touch sensor. Any “no touch” sensor, or proximity sensor, is contemplated to be used with embodiments of the sterilizer of the present invention. According to one embodiment, the no touch proximity sensor is a capacitive sensing wire looped around the circumference of the top and connected to capacitive sensing electronics. A change in capacitance sensed by the capacitive sensing wire and sensing electronics will activate the lid control mechanism to raise the lid and also activate the elevator mechanism to raise any items within the sterilizer for easy access and grabbing by a user who needs one of the items. According to another embodiment, the no touch proximity sensor could be an infrared (IR) sensor that can sense the heat of a person's hand as it approaches the lid. The items automatically lower into the sterilizer to be sanitized by UV light, and then raise up when the sterilization cycle completes or when proximity to an object, i.e., hand or finger, is sensed by the no touch proximity sensor.

Any items to be used with the sterilizer must be capable of fitting within one of the transparent tubes and resting on a floor within that tube. Of course, it will be understood that other embodiments of the sterilizer may be scaled to suit any particular application for any sized item. The transparent tubes are preferably configured to be transparent to UV light. According to one embodiment, the transparent tubes are quartz tubes.

According to one embodiment, the quartz tube may have a slot cut from a second end up toward, but not reaching, a first end of the tube to accommodate the elevator mechanism structure that supports the floors within the tubes. According to another embodiment, the quartz tube may have a slot that runs from the first end all the way to the second end of the tube, again to accommodate the elevator mechanism structure.

According to yet another embodiment, the inside surface of the cover of the sterilizer is highly reflective of UV light. According to still another embodiment, the inner surface of the cover is highly polished stainless steel to reflect UV light and clean all the surfaces of the items exposed to the UV light. According to another embodiment, the inner surface of the cover could be coated with a UV reflective paint or coating.

Timing of the lid opening and closing is carefully calibrated to avoid binding on the items during operation. According to one embodiment a cable is attached between the lid control and elevator mechanisms. The cable is given some slack in the open lid position. This allows the items to descend under control of the elevator mechanism to a safe location within the sterilizer prior to the slack in the cable being removed and the cable putting tension on the lid control mechanism and thereby closing the lid after the items have descended.

A torsion spring is used to open the lid. When the lid is closed, the cable is holding the lid down. Once the cable has some slack, it allows the torsion spring to open the lid. This torsion spring may also allow for some variation in the length of the cable. If the manufacturer makes the cable too short, this will stress the elevator mechanism. Conversely, if the manufacturer makes the cable too long, the lid will not close all the way. Upon opening, the cable may be under tension until the lid is completely open from lifting of the elevator mechanism. The cable then goes slack as the items rise to the full upright position for dispensing. An extension spring is connected between the cable and the lid control mechanism such that if the lid is closed and pried open without activating the elevator mechanism the spring stretches and allows the lid to open without breaking the cable or its attachments to the elevator and lid control mechanisms. The extension spring ensures that the lid closes tightly. According to one embodiment of the sterilizer, an electrical sensor prevents the UV light from turning on if the lid is open.

The following are technical features for embodiments of the sterilizer. Embodiments of the sterilizer use UV-C light to sterilize the writing implements. Note that the UV-C light will be completely concealed during a sterilization cycle, thereby avoiding unintended UV light exposure to skin and eyes. Embodiments of the sterilizer are configured to plug into a standard electrical outlet, for example a US outlet, a UK outlet, or a European outlet. Embodiments of the sterilizer are configured to fit easily on the front desk of a receptionist or other service provider where access to writing implements may be required. The shapes of various embodiments of the sterilizer are configured to be small, sleek and unobtrusive in a private or public office. Embodiments of the sterilizer are configured with a non-slip bottom. Embodiments of the sterilizer are configured to perform a sterilization cycle in 60 seconds. However, it will be understood that the UV light sterilization cycle could be configured to any suitable amount of time to perform its intended germicidal function, according to other embodiments. Embodiments of the sterilizer may be configured with up to four or six writing implements as illustrated herein. However, it will be understood that by scaling, embodiments of the sterilizer may be configured to any number of writing implement capacities. It will be understood that the term “sterilized” as used herein implies killing most all of the germs exposed to the UV light for a sufficient amount of time. It will also be understood that UV light sterilization will likely not apply to under pen cap or clip, since UV light may not impinge on those surfaces. Embodiments of the sterilizer may be configured for some variance in size and shape of the writing implements, for example and not by way of limitation, length up to 7″ and diameter up to ¾″. Embodiments of the sterilizer may be configured for easy and quick assembly.

The following are features of the user interface of the embodiments of the sterilizer as disclosed herein. Embodiments of the sterilizer may be configured for “no touch” operation by employing a proximity sensor near the lid. Embodiments of the sterilizer may be configured with a visible indicator showing that a sterilization cycle is underway. An embodiment of one such visible indicator may be a green light indicating that a sterilized writing implement is ready to use. Embodiments of the sterilizer may be configured to be small, having a high-tech appearance, unobtrusive, quiet, intuitive and easy to operate.

While the foregoing advantages of the present invention are manifested in the illustrated embodiments of the invention, a variety of changes can be made to the configuration, design and construction of the invention to achieve those advantages. Hence, reference herein to specific details of the structure and function of the present invention is by way of example only and not by way of limitation.

What is claimed is:

1. A sterilizer for sanitizing items, the sterilizer comprising:
a top including a lid, the lid configured for selectively covering a plurality of access ports;
a cover having an upper opening connected to the top and a lower opening;
a bottom connected to the lower opening of the cover;
wherein the space encapsulated by the top, the cover and the bottom contains:
at least one ultraviolet (UV) lamp configured to selectively illuminate the contents within the space;
a plurality of transparent tubes, each having a first and a second ends, the first end of each of the plurality of transparent tubes connected one of the plurality of access ports;
an elevator mechanism having floors within each of the plurality of transparent tubes accessed from the second ends of the transparent tubes, the elevator mechanism adapted for selectively raising and lowering any items placed within the plurality of transparent tubes through their respective access ports, the items resting on the floors; and
a lid control mechanism for selectively opening and closing the lid.

2. The sterilizer according to claim 1, wherein each of the plurality of transparent tubes is a quartz tube.

3. The sterilizer according to claim 2, wherein each of the plurality of quartz tubes has a slot cut from the second end up toward but not reaching the first end, the slot configured for an arm connected to one of the plurality of floors the arm further connected to the elevator mechanism.

4. The sterilizer according to claim 1, further comprising a no touch proximity sensor configured for automatically opening the lid upon sensing an object such as a finger or hand approaching the lid.

5. The sterilizer according to claim 4, wherein upon sensing an object approaching the lid, the lid automatically opens and the elevator mechanism lifts any items from within the sterilizer until they are at least partially extending from their respective access ports.

6. The sterilizer according to claim 1, further configured to receive one of the hand-held items placed through one of the plurality access ports and resting on one of the associated plurality of floors within its associated transparent tube with the lid open and automatically descend the floor along with the one item until the item is completely within the associated transparent tube followed by closing of the lid.

7. The sterilizer according to claim 1, further configured to illuminate the UV lamp for a preselected time, thereby sanitizing any items within the sterilizer.

8. The sterilizer according to claim 1, wherein the items are selected from the group consisting of: pens, pencils, highlighters and markers.

9. The sterilizer according to claim 1, wherein the lid is configured to open and close without binding on any items placed within the sterilizer.

10. The sterilizer according to claim 1, further comprising an extension spring in communication with the lid control mechanism for preventing breakage of the lid control mechanism if the lid is pried open.

11. The sterilizer according to claim 1, wherein the cover has a reflective internal surface to reflect UV rays emanating from the UV lamp and scattering the UV rays back toward contents of the sterilizer, particularly the items within the transparent tubes.

12. The sterilizer according to claim 1, further comprising an electrical sensor that prevents the at least one UV lamp from turning on if the lid is not closed.
